(19) United States
(12) Patent Application Publication
(10) Pub. No.: US 2013/0324585 A1
(43) Pub. Date: Dec. 5, 2013

(54) ROTIGOTINE IONIC LIQUID

(71) Applicant: Christian Janssen, Hannover (DE)

(72) Inventor: Christian Janssen, Hannover (DE)

(21) Appl. No.: 13/990,705

(22) PCT Filed: Nov. 29, 2011
(86) PCT No.: PCT/US11/62364

§ 371 Date: Aug. 19, 2013

Related U.S. Application Data
(60) Provisional application No. 61/419,070, filed on Dec. 2, 2010.

(51) Int. Cl. A61K 31/381

(52) U.S. Cl. CPC A61K 31/381
USPC 514/438; 549/75

(57) ABSTRACT

The present invention provides ionic liquids of rotigotine, pharmaceutical compositions comprising said ionic liquids and methods of preparing such ionic liquids. The invention further provides methods of using the compositions described herein to overcome problems arising from polymorphism, solubility and delivery, to control release rates, to add functionality, to enhance efficacy, and to improve ease of use and manufacture.

### CROSS REFERENCE TO RELATED APPLICATION

This patent application claims the benefit of U.S. provisional application No. 61/419,670, filed 2 Dec. 2010, the disclosure of which is herein incorporated by reference.

### FIELD OF THE INVENTION

The invention relates to ionic liquids of rotigotine, pharmaceutical compositions comprising said ionic liquids and methods of preparing such ionic liquids. The invention further relates to methods of using the compositions described herein to overcome problems arising from polymorphism, solubility and delivery, to control release rates, to add functionality, to enhance efficacy, and to improve ease of use and manufacture.

### BACKGROUND OF THE INVENTION

Aminotetralins constitute an important class of biologically active compounds. For example, the aminotetralin drug rotigotine ((S)-6-(propyl(2-thiophen-2-yl)ethylamino)-5,6,7,8 tetrahydronaphthalen-1-ol) is a non-ergot (or non-ergotamine) dopamine D2 agonist and is used for the treatment of Parkinson's disease and restless legs syndrome.

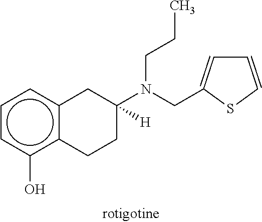

Rotigotine has been reported in at least two forms. U.S. Pat. No. 6,884,434 describes the HCl salt and the free base (rotigotine base). This patent also describes a pharmaceutical formulation of rotigotine in the form of a transdermal therapeutic system (transdermal patch) comprising an adhesive matrix layer containing rotigotine in an amount effective for the treatment of the symptoms of Parkinson's disease.

Rotigotine—especially in the form of its free base—is prone to oxidation as the structure bears a free phenolic hydroxyl group. Hence, every rotigotine form which is dedicated for a pharmaceutical formulation has to exhibit sufficient stability all the way through manufacturing, transport, storage and application.

The only rotigotine-containing drug formulation on the market is a transdermal patch for the treatment of Parkinson's disease which represents the first once-a-day transdermal patch to treat Parkinson's disease. Rotigotine is sold in Europe by UCB Pharma as Neupro®. It was approved in 2007 by the FDA for the U.S. In 2008, however, UCB Pharma had to recall all patches in the United States as the Rotigotine base crystallised from the transdermal patches.

Regardless of the crystallisation problem, Neupro®, which contains the free base of rotigotine, requires a continuous cold chain from the manufacturing, through storage, transport and distribution. Also for the patient, it is recommended to store Neupro® in the refrigerator.

The objective underlying this invention is to provide an advantageous form of rotigotine for pharmaceutical use, in particular in order to overcome or minimise the above mentioned problems.

This objective is solved by providing an ionic liquid of rotigotine, wherein the ionic liquid comprises at least one rotigotine cation and a counter ion derived from an organic compound or at least one rotigotine anion and a counter ion derived from an organic compound. Furthermore compositions are provided which comprise a rotigotine ionic liquid for pharmaceutical use.

It has been found by the inventors that rotigotine as a cation or as an anion can form ionic liquids with organic compounds that are easily prepared from a range of pharmaceutically acceptable organic compounds. These ionic rotigotine liquids show good thermal stability. In particular they overcome the problems with crystallisation which exists for the free base as well as the solid salts known in the prior art.

The ionic liquids according to the present invention exhibit a melting point below 40° C., preferably a melting point below 32° C.

In the ionic liquid of rotigotine of the present invention comprising at least one rotigotine cation and a counter ion derived from an organic compound, the counter ion derived from the organic compound is a counter ion that leads to the formation of the ionic liquid of rotigotine exhibiting a melting point below 40° C., preferably a melting point below 32° C.

In the ionic liquid of rotigotine of the present invention comprising at least one rotigotine anion and a counter ion derived from an organic compound, the counter ion derived from the organic compound is a counter ion that leads to the formation of the ionic liquid of rotigotine exhibiting a melting point below 40° C., preferably a melting point below 32° C.

The ionic liquids of rotigotine according to the invention thus have the advantage that problems related to crystalline polymorphism do not arise. Pharmaceuticals which exist in polymorphic forms often reveal the problem of physical instability when they are stored in pharmaceutical preparations. Since changes of the crystalline form can have tremendous effects on the physicochemical parameters of a substance, avoidance of the solid state gives a clear benefit.

The ionic liquids disclosed herein are composed of at least a rotigotine cation and at least one kind of anion or, alternatively of at least a rotigotine anion and at least one kind of cation. It is contemplated that the disclosed ionic liquids can comprise a rotigotine cation with more than one kind of anion (e.g., 2, 3 or more different kinds of anions). Preferred is one anion. Likewise, it is contemplated that the disclosed ionic liquids can comprise a rotigotine anion with more than one kind of cation (e.g., 2, 3 or more different kinds of cations). Preferred is one cation. The ions preferably are pharmaceutically acceptable.

In a preferred embodiment of the invention the stoichiometry of the rotigotine ion:counter ion in the ionic liquid is between 1:3 and 1:1, preferably 1:2 or 1:1 and most preferably near 1:1.

In yet another embodiment of the invention the rotigotine ionic liquid comprises a rotigotine cation and at least 50% of the anions deriving from an organic acid. Accordingly, a composition comprising rotigotine salts with different anions derived from organic acids and inorganic acids such as HCl, form part of the invention, if at least 50% of the rotigotine anions are derived from the organic acid.

In a further aspect of the invention the organic compound from which the counter ion is derived is of lipophilic nature, which is defined by a partition coefficient log P value of more than 0. Preferably, said lipophilic compound has a log P value of >1, more preferably of more than 2, even more preferably of more than 3, and most preferably of more than 4.

In one aspect of the invention the counter ion is defined as an organic compound exhibiting the following characteristics:

1. Partition coefficient log P in a range between 0 and 6

2. Molecular weight from 100 to 500

3. Number of atoms from 20 to 70

In another embodiment, the invention relates to a composition which comprises a rotigotine salt, wherein at least 1%, preferably at least 2% and more preferably at least 5% of the salt is an ionic liquid. Accordingly the composition can comprise, in addition to the rotigotine ionic liquid, rotigotine salts, e.g., solid salts, with different anions which are not ionic liquids. In such compositions according to the invention, the rotigotine ionic liquid thus amounts to at least 1%, preferably at least 2% or at least 5% of the total rotigotine salt in such composition.

Cations as Counter-Ions

In one aspect of the invention the ionic liquid of rotigotine comprises a rotigotine anion and a cationic counter ion selected from the group of cations defined as follows:

Quaternary Ammonium Compounds (QACs)

Particular examples of cationic compounds that can be present in the disclosed ionic liquids are compounds that contain nitrogen atoms. Nitrogen atoms can exist or can be converted to positively-charged quaternary ammonium species, for example, through alkylation or protonation of the nitrogen atom. Thus, compounds that possess a quaternary nitrogen atom (known as quaternary ammonium compounds (QACs)) are typically cations. According to the methods and compositions disclosed herein, any compound that contains a quaternary nitrogen atom or a nitrogen atom that can be converted into a quaternary nitrogen atom can be a suitable cation for the disclosed ionic liquids.

According to the invention, the ionic liquid comprises a rotigotine anion and at least one quaternary amine of the formula (I):

R¹R²R³R⁴N⁺ (I)

wherein R¹, R², R³ and R⁴ are each independently H,

optionally substituted C₁ to C₅ alkyl or alkenyl

optionally substituted C₆ to C₁₀ cycloalkyl,

optionally substituted C₆ to C₁₂ aryl

optionally substituted C7 to C12 alkaryl,

or wherein R¹ and R² taken together represent a C₄ to C₁₀ optionally substituted alkylene group, thereby forming with the N atom of formula (I) a 5 to 11-membered heterocyclic ring, and wherein the term “optionally substituted” means that the group in question may or may not be substituted with one or more groups (preferably from 0 to 6 groups) selected from OH, SH, SR⁵, Cl, Br, F, I, NH₂, CN, NO₂, COOR⁵, CHO, COR⁵ and OR⁵, wherein R⁵ is a C₁ to C₁₀ alkyl or cycloalkyl group.

Tetraalkyl Ammonium

In one embodiment of the invention the cationic counter ion is a tetraalkyl ammonium cation. In one example, a tetraalkyl ammonium cation can comprise one long chain alkyl moiety (e.g., 10 or more carbon atoms in length) and three short chain alkyl moieties (e.g., less than 10 carbon atoms in length).

The disclosed ionic liquids can also comprise an aliphatic benzyl alkyl ammonium cation. An aliphatic benzylalkyl ammonium cation is a cation that comprises an aliphatic moiety bonded to the nitrogen atom of a benzyl alkyl amine moiety. The aliphatic moiety can be as described herein. The benzylalkyl amine moiety can be a benzyl amine where the amine is bonded to an alkyl or cyclic alkyl group, as described herein, One or more types of aliphatic benzylalkyl ammonium cation can be used in the ionic liquids disclosed herein,

In one aspect, the aliphatic benzylalkyl ammonium cation can be represented by the following formula:

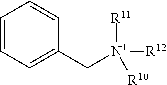

wherein R¹⁰ is an aliphatic group, as described above, R¹¹ and R¹² are, independent of one another, alkyl groups or cyclic alkyl groups as described herein, In some examples, one or more of the “R” substituents can be a long chain alkyl group (i.e. the number of carbon atoms is 10 or greater). In other examples, one or more of the “R” substituents can be a short chain alkyl group (i.e. the number of carbon atoms is less than 10), In still other examples, one of the “R” substituents is a long chain alkyl group and the other two “R” substituents are short chain alkyl groups.

In one aspect, the aliphatic benzylalkyl ammonium cation can have any of the aliphatic moieties disclosed herein bonded to any benzyl alkyl amine moieties disclosed herein, In some specific examples, R¹⁰ in the formula of aliphatic benzylalkyl ammonium cation can be an aliphatic group of from 10 to 40 carbon atoms, e.g. a decyl, dodecyl (lauryl), tetradecyl (myristyl), hexadecyl (palmityl or cetyl), octadecyl (stearyl), or eicosyl (arachidyl) group, and R¹¹ and R¹² can each be, independent of one another, a methyl, ethyl, propyl, butyl, pentyl, or hexyl group.

In another aspect, the aliphatic benzylalkyl ammonium cation can include, but are not limited to, alkyl dimethyl benzyl ammonium cations, Specific examples of alkyl dimethyl benzyl ammonium cations include, but are not limited to, cetyl dimethyl benzyl ammonium, lauryl dimethyl benzyl ammonium, myristyl dimethyl benzyl ammonium, stearyl dimethyl benzyl ammonium, and arachidyl dimethyl benzyl ammonium.

In yet another aspect, the aliphatic benzylalkyl ammonium cation can include, but are not limited to, alkyl methyl ethyl benzyl ammonium cations. Specific examples of alkyl methyl ethyl benzyl ammonium cations include, but are not limited to, cetyl methyl ethyl benzyl ammonium, lauryl methylethyl benzyl ammonium, myristyl methylethyl benzyl ammonium, stearyl methyl ethyl benzyl ammonium, and arachidyl methylethyl benzyl ammonium.

Dialiphatic Dialkyl Ammonium

Still further examples of QACs that can be used in the disclosed ionic liquids are dialiphatic dialkyl ammonium cations. A dialiphatic dialkyl ammonium cation is a compound that comprises two aliphatic moieties and two alkyl moieties bonded to a nitrogen atom. The aliphatic moieties can be the same or different and can be any aliphatic group as described above. The alkyl moieties can be the same or different can be any alkyl group as described above. In the disclosed dialiphatic dialkyl ammoniums cations, the two aliphatic moieties can have 10 or more carbon atoms and the two alkyl moieties can have less than 10 carbon atoms. In another alternative, the two aliphatic moieties can have less than 10 carbon atoms and the two alkyl moieties can have 10 or more carbon atoms. One or more types of dialiphatic dialkyl ammonium cations can be used in the ionic liquids disclosed herein.

In some particular examples, the dialiphatic dialkyl ammonium cation can be didodecyl dimethyl ammonium, di-tetradecyl dimethyl ammonium, dihexadecyl dimethyl ammonium, and the like, including combinations thereof.

Other Cations

In a particular embodiment of the invention choline esters (e.g. acetylcholine) can be used as a counter ion. Choline esters can be provided by esterifying a compound containing a carboxylic acid moiety or transesterifying a compound with an ester moiety with a choline moiety. Further examples of choline esters are bethanechol, carbachol, citocoline, methacholine, succinylmonocholine, suxamethonium chloride. In a specific aspect of the invention choline can be used as a counter ion.

Further examples of preferred cations are (2-hydroxyethyl)dimethylundecyloxymethyl-ammonium, (2-acetoxyethyl)heptyloxymethyldimethylammonium, and (2-acetoxyethyl)dodecyloxymethyldimethylammonium and mepenzolate.

In one embodiment of the invention the cationic counter ion derives from an arylamine.

In a further embodiment of the invention the cationic counter ion derives from an alkylamine, which is preferably ethylenamine or piperazine.

In another embodiment of the invention the cationic counter ion derives from an alkylamine comprising hydroxy groups, which is preferably diethanolamine, triethanolamine, tromethamine or N-methylglucamine.

In a further preferred embodiment ionic liquids of rotigotine are provided using amines bases with high lipophilicity such as benzathine, 4-phenylcyclohexylamine, benethamine, and hydrabamine.

In the most preferred embodiment of the invention the ionic liquid comprises a rotigotine anion and a N,N,N-trimethylethanolammonium cation (choline).

Anions as Counter Ions

In a further aspect of the invention an organic acid is used for the preparation of the ionic liquid. In a preferred embodiment said organic acid has the formula R—COOH, wherein R is a saturated and mono-unsaturated, branched or unbranched C₃-C₁₆ hydrocarbyl residue. This hydrocarbyl residue preferably has 3 to 16 carbon atoms, more preferably 5 to 16 carbon atoms. In a further embodiment, R is preferably a saturated, mono-unsaturated or poly-unsaturated, unbranched C₃-C₁₆ hydrocarbyl, more preferably a C₅ to C₁₆ hydrocarbyl.

Examples of preferred acids of the formula R—COOH are propionic acid, butyric acid, pentanoic acid, hexanoic acid, heptanoic acid, octanoic acid, nonanoic acid, decanoic acid, undecanoic acid, lauric acid, tridecanoic acid, myristic acid, pentadecanoic acid and palmitic acid. Particularly preferred is octanoic acid.

In yet another aspect of the invention the organic acid has the formula R′—COOH, wherein R′ is a polyunsaturated, branched or unbranched, C₁₈-C₂₂ hydrocarbyl residue.

Examples of preferred acids of the formula R′—COOH are linoleic acid, alpha-linoleic acid, arachidonic acid, eicosapentaenoic acid and docosahexaenoic acid. Particularly preferred is linoleic acid.

Other suitable anions contemplated herein are derived from long-chain alkylsulfonic acids (e.g. lauryl sulfonic acid), saccharine or acesulfame.

In one embodiment of the invention the anionic counter ion is derived from a lipophilic monovalent acid. Preferably the counter ion derived from the lipophilic monovalent acid represents at least 50% of the anions within the ionic liquid. The lipophilic monovalent acid preferably is octanoic acid.

Methods for the Preparation of Ionic Liquids

General Methods of Preparation

There are generally three methods for preparing an ionic liquid:

(1) Metathesis of a salt of the desired cation (e.g., a halide salt) with a salt of the desired anion (e.g., transition metal, like Ag, salt, Group I or II metal salt, or ammonium salt). Such reactions can be performed with many different types of salts; and

(2) An acid-base neutralization reaction.

(3) Displacing the relevant ion from a salt by addition of a stronger base or acid

Combining rotigotine ion with a counter-cation in a solvent with optional heating can thus produce the ionic liquids.

Preparation of a Rotigotine Ionic Liquid Using an Organic Acid

The ionic liquid of the present invention of rotigotine and an organic acid as the anion forming agent can be prepared by mixing rotigotine and the acid and recovering the ionic liquid. In one embodiment, the mixing step is carried out in the absence of any solvent which has the advantage that it is not necessary to remove any solvent after the ionic liquid forming reaction and additionally makes the process environmentally friendly. Yet another advantage of preparing the ionic liquid in the absence of a solvent is that the resulting ionic liquid does not contain any residual solvents.

Alternatively, the rotigotine and the organic acid can be mixed in the presence of one or more solvents. In this case, either the rotigotine or the acid or the rotigotine and the acid can be suspended or dissolved in the same or different solvents before mixing. For example, first the rotigotine can be dissolved in the solvent and then the acid is added to this solution. Suitable solvents are, e.g., water, alcohols, ether, ketones, chlorinated solvents, and mixtures thereof. Preferred solvents are ethers and ketones.

Preparation of a Rotigotine Ionic Liquid Using an Amine

The ionic liquid of the present invention of rotigotine and an amine as the cation-forming agent can be prepared by mixing rotigotine or a suitable acid-addition salt of rotigotine and the corresponding amine and optionally an additional base in the presence of one or more solvents. In this case, either the rotigotine, the amine and/or the optional additional base can be suspended or dissolved in the same or different solvents before mixing. For example, rotigotine can be dissolved in the solvent and then the amine and the optional additional base is added to this solution. Suitable solvents are, e.g. water, alcohols, ethers, ketones, chlorinated solvents, and mixtures thereof. Preferred solvents are alcohols. The optional additional base is a strong base such as hydroxides or hydrides. Preferred optional additional bases are sodium hydroxide and potassium hydroxide.

Pharmaceutical Use of the Ionic Liquids

In one embodiment of the invention the ionic liquid can be used as “neat ionic liquid”, that is, that the ionic liquid is substantially constituted by the cations and anions forming the ionic liquid. It is understood, that such neat ionic liquids can comprise additional materials or impurities. The neat ionic liquid according to the invention thus is defined as containing at least 85%, preferably at least 90%, more preferably at least 95%, even more preferably at least 98 or at least 99% and specifically at least 99% cations and anions forming the ionic liquid. As an additional material the neat ionic liquid may comprise solvent molecules (e.g., water). However, these solvent molecules should not be present in excess in the sense that the disclosed ionic liquids are dissolved in the solvent, forming a solution. That is, the disclosed ionic liquids contain no or minimal amounts of solvent molecules that are free and not bound or associated with the ions present in the ionic liquids. Thus, the disclosed ionic liquids can be liquid hydrates or solvates, but not solutions.

In one embodiment of the invention the neat ionic liquid is substantially free of water. By substantially free is meant that water is present at less than about 15 wt. %, preferably less than about 5 wt. %, more preferably less than about 2 wt. %, based on the total weight of the ionic liquid.

In addition, an ionic liquid with higher viscosity can be administered as an oil or cream for a topical administration. In a further embodiment, the ionic liquids of the present invention can be used for depot injections.

The neat ionic liquid as such can constitute a pharmaceutical composition. This is the case, when the additional material which can be present in the neat ionic liquid represents an excipient, such as e.g. water.

In a further aspect of the invention the ionic liquids are part of a composition, in particular a pharmaceutical composition. A pharmaceutical composition of the invention comprises at least an ionic liquid of rotigotine and a pharmaceutically acceptable carrier. The selection of acceptable carriers and the formulation of pharmaceutical compositions is generally known to the skilled person.

Such pharmaceutically acceptable carriers, most typically, are e.g. sterile water, saline, and buffered solutions at physiological pH (preferably from about 5 to about 8, and more preferably from about 7 to about 7.5). Further examples of pharmaceutically acceptable carriers include, but are not limited to, Ringer's solution and dextrose solution.

Pharmaceutical compositions can include additional carriers, as well as thickeners, solvents, diluents, buffers, preservatives, dyes, colorants, viscosity modifiers, surface active agents, mixtures and combinations thereof and the like in addition to the compounds disclosed herein.

It is understood, however, that the disclosed ionic liquids can, though need not, be solubilised, and solutions of the disclosed ionic liquids are contemplated herein. Further, the disclosed ionic liquids can be formulated in an extended or controlled release vehicle, for example, by encapsulating the ionic liquids in microspheres or microcapsules using methods known in the art. Still further, the disclosed ionic liquids can themselves be solvents for other solutes. For example, the disclosed ionic liquids can be used to dissolve a particular non-ionic or ionic pharmaceutical active.

In one preferred aspect the invention provides a method of manufacturing a transdermal delivery system comprising the rotigotine ionic liquids as disclosed herein and such transdermal delivery system. In general, means of making rotigotine transdermal patches are disclosed in U.S. Pat. No. 6,929,801, which is incorporated by reference in its entirety herein.

### DEFINITIONS

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the term “pharmaceutically acceptable” refers to a compound that is generally safe and non-toxic for humans and/or animals.

The term “ionic liquid” is used herein to refer to salts (i.e., compositions comprising organic cations and organic anions) that exhibit a melting point below 40° C., preferably a melting point below 32° C.

The present invention will now be further illustrated by the following examples, which are not intended to be construed as limiting.

### EXAMPLE 1

### Ionic Liquid Containing a Rotigotine Cation and an Octanoic Anion

The reaction equation for preparation of rotigotine octanoate is given in the following:

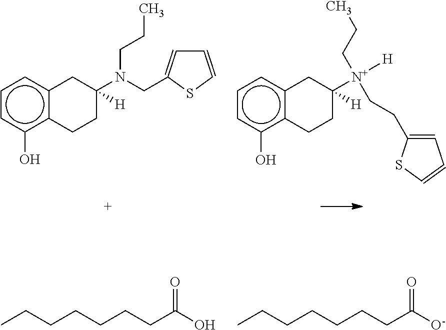

### EXAMPLE 1a

To a solution of rotigotine (1 eq) in diisopropyl ether (5 volumes) was added octanoic acid (1 eq) under nitrogen. After stirring for 1 h, the solvent was removed in vacuo at 30° C. Drying on the rotary evaporator was continued for another 2 h at this temperature. Drying in high vacuum for 2 h yielded an oil in quantitative yield.

### EXAMPLE 1b

Rotigotine (1 eq) was added to octanoic acid (1 eq) in a glass vial. The mixture was shaken for 1 h to form a homogenous oil.

### EXAMPLE 2

### Ionic Liquid Containing a Rotigotine Anion and a Choline Cation

The reaction equation for preparation of the rotigotine choline ionic liquid is given in the following:

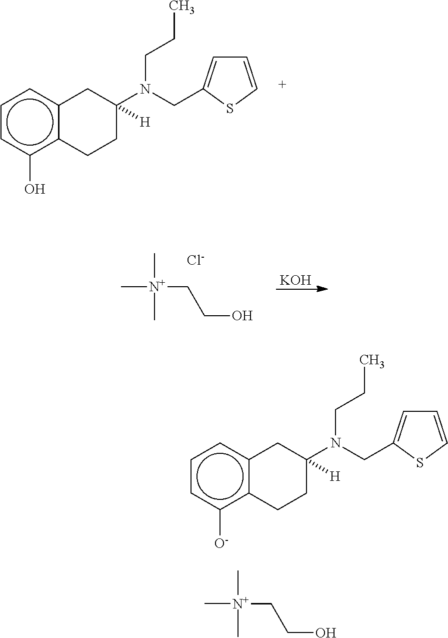

### EXAMPLE 2a

Rotigotine (1 eq) is dissolved in methanol (5 volumes) in a round bottom flask. Potassium hydroxide (1 eq) and choline chloride (1 eq) are added and the mixture is stirred at room temperature for 1 h. Acetone (5 volumes) is added and the mixture is filtered through a fine filter. The filtrate is concentrated in vacuo at room temperature and dried using high vacuum. The product is obtained as an oily residue in quantitative yields.

### EXAMPLE 2b

Rotigotine hydrochloride (1 eq) is dissolved in methanol (5 volumes) in a round bottom flask. Potassium hydroxide (2 eq) and choline chloride (1 eq) are added and the mixture is stirred at room temperature for 1 h. Acetone (5 volumes) is added and the mixture is filtered through a fine filter. The filtrate is concentrated in vacuo at room temperature and dried using high vacuum. The product is obtained as an oily residue in quantitative yields.

1. Ionic liquid of rotigotine, wherein the ionic liquid comprises (a) at least one rotigotine cation and at least one counter ion derived from an organic compound, or (b) at least one rotigotine anion and at least one counter ion derived from an organic compound.

2. Ionic liquid according to claim 1, comprising the at least one rotigotine cation and the at least one counter ion derived from an organic compound, wherein the at least one counter ion derived from the organic compound is at least one counter ion that leads to the formation of the ionic liquid of rotigotine exhibiting a melting point below 40° C., preferably a melting point below 32° C.

3. Ionic liquid according to claim 1, comprising the at least one rotigotine anion and the at least one counter ion derived from an organic compound, wherein the at least one counter ion derived from the organic compound is at least one counter ion that leads to the formation of the ionic liquid of rotigotine exhibiting a melting point below 40° C., preferably a melting point below 32° C.

4. Ionic liquid according to one of claims 1 to 3, wherein the organic compound is lipophilic.

5. Ionic liquid according to claim 2, wherein the ionic liquid comprises the at least one rotigotine cation and the at least one counter ion wherein the at least one counter ion is at least an anion derived from at least one organic acid.

6. Ionic liquid according to claim 5, wherein the at least one organic acid is selected from:
(i) a compound of the formula R—COOH, wherein R is a saturated or monounsaturated, branched or unbranched C₃-C₁₆ hydrocarbyl residue or
(ii) a compound of the formula R′—COOH, wherein R′ is a polyunsaturated, branched or unbranched C₁₈-C₂₂ hydrocarbyl residue.

7. Ionic liquid according to any one of claims 5 and 6, wherein the at least one organic acid is selected from propionic acid, butyric acid, pentanoic acid, hexanoic acid, heptanoic acid, octanoic acid, nonanoic acid, decanoic acid, undecanoic acid, lauric acid, tridecanoic acid, myristic acid, pentadecanoic acid, palmitic acid, unsaturated analogs thereof, linoleic acid, alpha-linoleic acid, arachidonic acid, eicosapentaenoic acid and docosahexaenoic acid.

8. Ionic liquid according to claim 7, wherein the at least one organic acid is octanoic acid.

9. Ionic liquid according to claim 7, wherein the at least one organic acid is linoleic acid.

10. Ionic liquid according to claim 3, wherein the ionic liquid comprises the at least one rotigotine anion and the at least one counter ion, and wherein the at least one counter ion is at least one cation derived from at least one amine.

11. Ionic liquid according claim 10, wherein said at least one amine cation is selected from:
(i) an amine of the formula (I):
R¹R²R³R⁴N⁺ (I)
wherein R¹, R², R³ and R⁴ are each independently H,
optionally substituted C₁ to C₅ alkyl or alkenyl
optionally substituted C₆ to C₁₀ cycloalkyl,
optionally substituted C₆ to C₁₂ aryl, or
optionally substituted C₇ to C₁₂ alkaryl,
or wherein R¹ and R² taken together represent a C₄ to C₁₀ optionally substituted alkylene group, thereby forming with the N atom of formula (I) a 5 to 11-membered heterocyclic ring, and wherein the term “optionally substituted” means that the group in question may or may not be substituted with one or more groups, preferably from 0 to 6 groups, selected from OH, SH, SR⁵, Cl, Br, F, I, NH₂, CN, NO₂, COOR⁵, CHO, COR⁵ and OR⁵, wherein R⁵ is a C₁ to C₁₀ alkyl or cycloalkyl group,
(ii) a tetraalkyl ammonium ion; or
(iv) an aliphatic heteroaryl ammonium ion; or
(v) a dialiphatic dialkyl ammonium cation.

12. Ionic liquid according to claim 10, wherein the at least one amine is selected from diethanolamine, triethanolamine, ethylenamine, trimethamine, N-methylglucamine, piperazine, benzathine, 4-phenylcyclohexylamine, benethamine, and hydrabamine.

13. Ionic liquid according to claim 10, wherein the at least one counter ion is N,N,N-trimethylethanolammonium cation.

14. A method for the treatment, prevention, amelioration, or elimination of a disease that is susceptible to the administration of a dopamine D2 receptor agonist or an antiparkinson agent said method comprising administering an effective amount of the ionic liquid according to any one of claims 1 to 3 in a subject in need thereof.

15. The method according to claim 14, wherein the disease is Parkinson's disease, restless legs syndrome (RLS), or intermittent RLS.

16. Pharmaceutical composition comprising a rotigotine salt, wherein at least 1 wt. %, preferably at least 2 wt. % and more preferably at least 5 wt. % of the salt is an ionic liquid as defined according to one of claims 1 to 3 and a pharmaceutically acceptable carrier.

17. A transdermal delivery system comprising a rotigotine salt, wherein at least 1 wt. %, preferably at least 2 wt. %, and more preferably at least 5 wt. %, of the salt is an ionic liquid as defined according to one of claims 1 to 3.

18. Method of preparing an ionic liquid according to any one of the claims 1, 2, 5 and 6, comprising the following steps:
(i) mixing rotigotine base and at least one organic acid, optionally in the presence of a solvent;
(ii) optionally removing the solvent if the solvent is used in step (i); and
(iii) recovering the ionic liquid.

19. Method of preparing an ionic liquid according to any one of claims 1, 3 and 10 to 13, comprising the following steps:
(i) mixing rotigotine or an acid-addition salt of rotigotine and at least one amine and optionally an additional base in the presence of one or more solvents;
(ii) optionally removing the solvent(s) if the solvent(s) is used in step (i); and
(iii) recovering the ionic liquid.

20. Pharmaceutical composition comprising a rotigotine salt, wherein at least 2 wt. % of the salt is an ionic liquid as defined according to one of claims 1 to 3 and a pharmaceutically acceptable carrier.

21. A transdermal delivery system comprising a rotigotine salt, wherein at least 2 wt. % of the salt is an ionic liquid as defined according to one of claims 1 to 3.

22. Pharmaceutical composition comprising a rotigotine salt, wherein at least 5 wt. % of the salt is an ionic liquid as defined according to one of claims 1 to 3 and a pharmaceutically acceptable carrier.

23. A transdermal delivery system comprising a rotigotine salt, wherein at least 5 wt. % of the salt is an ionic liquid as defined according to one of claims 1 to 3.

24. Method of preparing an ionic liquid according to claim 1 or 2, comprising the following steps:
(i) mixing rotigotine base and at least one organic acid, in the presence of a solvent;
(ii) removing the solvent; and
(iii) recovering the ionic liquid

25. Method of preparing an ionic liquid according to any one of claims 1 and 3, comprising the following steps:
(i) mixing rotigotine or an acid-addition salt of rotigotine, at least one amine and an additional base in the presence of one or more solvents;
(ii) removing the solvent(s); and
(iii) recovering the ionic liquid.

26. Ionic liquid according to claim 4, wherein the ionic liquid comprises the at least one rotigotine cation and the at least one counter ion wherein the at least one counter ion is at least an anion derived from at least one organic acid.

27. Ionic liquid according to claim 26, wherein the at least one organic acid is selected from:
(i) a compound of the formula R—COOH, wherein R is a saturated or monounsaturated, branched or unbranched C₃-C₁₆ hydrocarbyl residue or
(ii) a compound of the formula R′—COOH, wherein R′ is a polyunsaturated, branched or unbranched C₁₈-C₂₂ hydrocarbyl residue.

28. Ionic liquid according claim 27, wherein the at least one organic acid is selected from propionic acid, butyric acid, pentanoic acid, hexanoic acid, heptanoic acid, octanoic acid, nonanoic acid, decanoic acid, undecanoic acid, lauric acid, tridecanoic acid, myristic acid, pentadecanoic acid, palmitic acid, unsaturated analogs thereof, linoleic acid, alpha-linoleic acid, arachidonic acid, eicosapentaenoic acid and docosahexaenoic acid.

29. Ionic liquid according to claim 28, wherein the at least one organic acid is octanoic acid or linoleic acid.
